(19) United States
(12) Patent Application Publication
(10) Pub. No.: US 2011/0014243 A1
(43) Pub. Date: Jan. 20, 2011

(54) PHARMACEUTICAL COMPOSITIONS

(76) Inventors: Carl-Magnus Andersson, Hjarup (SE); Johanna Qvarnstrom, Huddinge (SE); Jonas Berggren, Uppsala (SE); Marita Nilsson, Grodinge (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 12/735,018

(22) PCT Filed: Dec. 15, 2008
(86) PCT No.: PCT/EP2008/010636

§ 371 Date: Sep. 3, 2010

(30) Foreign Application Priority Data
Dec. 14, 2007 (GB) 07 24478.3

(51) Int. Cl. A61K 31/196; A61K 9/00; A61P 3/06; A61P 3/10; A61P 25/24; A61P 19/08; A61P 3/04

(52) U.S. Cl. 424/400; 514/563

(57) ABSTRACT

Compositions are described in which certain thyroid hormone receptor-binding compounds are formulated together with a basic filler. Such formulation acts to prevent the formation of undesired reaction products. The compositions may be formed into granules or a tablet which may optionally be enterically coated.

This invention relates to pharmaceutical compositions. In particular, it relates to pharmaceutical compositions in which the pharmacologically-active ingredients are protected from degradation.

Compound 1A having the following structure:

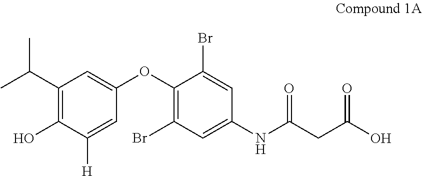

is described in WO 01/60784 (IUPAC name 3-[[3,5-Dibromo-4-[4-hydroxy-3-(1-methylethyl)-phenoxy]-phenyl]-amino]-3-oxopropanoic acid). Compound 1A and a series of related compounds are described as agonists of thyroid hormone receptors, in particular the TRβ receptor. Such compounds should be useful in the treatment or prevention of a disease associated with metabolic dysfunction or which is dependent upon the expression of a triiodothyronine (T₃)-regulated gene. Such diseases include, for example, obesity, hypercholesterolemia, atherosclerosis, cardiac arrhythmias, depression, osteoporosis, hypothyroidism, goitre, thyroid cancer as well as glaucoma and congestive heart failure. Formulations of Compound 1A and related compounds are disclosed in WO 2007/110226.

It has been found that certain compositions containing Compound 1A tend to discolour on prolonged storage indicating degradation of the composition over extended periods. Furthermore, it has been found that significant degradation of the compositions, specifically, degradation of the active ingredient, Compound 1A, occurs rapidly when the compositions are stored at room temperature and refrigeration is necessary if the compositions are to be stored without unacceptable levels of degradation occurring.

According to a first aspect, the present invention provides a pharmaceutical composition suitable for oral administration, comprising an admixture of:
(i) a compound of Formula I:

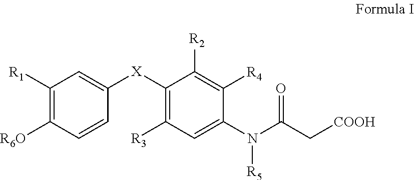

wherein:
R₁ is selected from hydrogen, halogen, trifluoromethyl, C₁₋₆ alkyl, or C₃₋₇ cycloalkyl;
X is oxygen (—O—), or methylene (—CH₂—);
R₂ and R₃ are the same or different and are halogen or C₁₋₄ alkyl;
R₄ is hydrogen or C₁₋₄ alkyl;
R₅ is hydrogen or C₁₋₄ alkyl; and
R₆ is hydrogen, or an alkanoyl or aroyl group, or other group capable of bioconversion to generate the free phenol structure wherein R₆=H;
   or a pharmaceutically acceptable salt or ester or solvate thereof;
(ii) a pharmaceutically acceptable basic particulate solid; and
(iii) optionally one or more further pharmaceutical excipients,
   wherein component (ii) makes up at least 1% by weight of the admixture.

Preferably, the compound of Formula I is Compound 1A or a pharmaceutically acceptable salt or ester or solvate thereof.

It was surprisingly found that a composition comprising an admixture of a compound of Formula I and at least 1% by weight based on the total weight of the admixture of a pharmaceutically acceptable basic particulate solid, such as calcium carbonate, significantly reduced the degradation of the composition as compared to formulations not comprising such basic particulate solid, particularly compositions such as those disclosed in WO 2007/110226. Compositions comprising compounds of Formula I admixed with calcium carbonate were found to have enhanced shelf stability, especially over extended periods and/or when stored at room temperature. In contrast various other pharmaceutical excipients such as mannitol had a detrimental effect on the stability of the compositions comprising compounds of Formula I. In particular, it has been found that even compounds of Formula I that have been milled during the preparation of a dosage form do not significantly degrade when the pharmaceutical composition includes a pharmaceutically acceptable basic particulate solid, such as calcium carbonate. Degradation has proved to be a significant problem in milled compositions. The term “milled” as employed herein refers to the grinding of a solid in a mill or other grinder to form a fine powder.

A major degradation product of Compound 1A has been found to arise from decarboxylation of the β-keto carboxylic acid functional group to form an acetamide (Compound 2A). It was found that when a pharmaceutical composition comprising Compound 1A also comprises calcium carbonate the formation of Compound 2A is significantly slowed.

The term “basic particulate solid” as used herein refers to a particulate solid which provides an alkaline pH in pure water. The particulate solid may provide an alkaline pH when dispersed or dissolved in pure water. Preferably, 10% by weight of the basic particulate solid in pure water provides a pH of greater than 7.0, more preferably a pH of at least 7.5, and especially a pH of at least 8.0. Preferably, 10% by weight of the basic particulate solid in pure water provides a pH of at least 8.0, preferably from 8.0 to 12.0, especially from 8.0 to 10.0. The term “acidic” used herein relates to a substance which provides an acidic pH in water. For example, a substance that provides a pH of less than 7.0, preferably a pH of less than 6.5 and especially a pH of less than 6.0 when dispersed or dissolved in pure water. A neutral substance is a substance that provides a neutral pH in water, that is, a substance that provides a pH of approximately 7.0 when dispersed or dissolved in pure water. The pharmaceutically acceptable basic particulate solid may for example be an alkali metal (Group 1) or alkaline earth metal (Group 2) salt. The corresponding anion may for example be an anion derived from an acid having a pKa value higher than 3.6. The term “filler” as used herein relates to a particulate solid material used to make up the bulk of a pharmaceutical preparation.

Preferred basic particulate solids include Group 1 and Group 2 metal carbonates, bicarbonates and carboxylates, including for example acetates and citrates. Certain sulfates, phosphates and hydrogen phosphates may also be used. Preferably, the basic particulate solid is a Group 1 or Group 2 metal carbonate or bicarbonate. Examples of suitable basic particulate solids include sodium carbonate, potassium carbonate, magnesium carbonate, calcium carbonate, sodium bicarbonate, potassium bicarbonate, magnesium bicarbonate and calcium bicarbonate. Additional basic particulate solids include basic clays, for example attapulgite; talc; calcium sulfate; basic silicates such as magnesium aluminium silicate; basic oxides such as magnesium oxide; sodium or potassium citrate; and basic starches, for example maize starch. Other examples of suitable pharmaceutically acceptable basic particulate solids are listed in the Handbook of Pharmaceutical Excipients, 5th edition (2006), published by McGraw-Hill Medical, New York, USA. Particularly preferred basic particulate solids include calcium carbonate and magnesium carbonate. A 10% by weight dispersion of calcium carbonate in pure water provides a pH of approximately 8.3. The composition may include more than one basic particulate solid. Pharmaceutically acceptable basic particulate solids are present at a level sufficient to attenuate the degradation of the compositions. The specific level which is sufficient to achieve such attenuation is, in part at least, dependent on the overall composition of the tablet or other solid dosage form. In certain matrices, a low loading sufficient to give an excess of basic particulate solid over the compound of formula I, such as Compound 1A, will suffice, however the use of higher levels of the basic particulate solid are preferred. Oral dosage forms comprising from 1% are included in the present invention. Preferably, pharmaceutically acceptable basic particulate solids make up at least 5%, for example at least 10%, for example at least 20%, for example at least 40%, for example at least 50%, for example at least 60%, for example at least 70% by weight of the admixture. In some embodiments, pharmaceutically acceptable basic particulate solids make up at least 80% by weight of the admixture. In other embodiments, pharmaceutically acceptable basic particulate solids make up from 10 to 20% by weight of the admixture. In embodiments in which the composition comprises further components such as a coating in addition to the admixture, the pharmaceutically acceptable basic particulate solids preferably make up at least 10% by weight, 20% by weight, for example at least 40%, for example at least 50%, for example at least 60% by weight of the entire composition. In some embodiments in which the composition comprises further components in addition to the admixture, pharmaceutically acceptable basic particulate solids make up at least 70% by weight of the entire composition, for example at least 80% of the entire composition. Preferably the basic particulate solid is present in the composition as a filler. Fillers other than pharmaceutically acceptable basic particulate solids may be present if desired. In one embodiment of the invention, the composition, and in particular the admixture comprising the compound of Formula I or salt or ester or solvate thereof, is substantially free of any fillers other than basic particulate solids, for example acidic or neutral fillers. In another embodiment, the composition and especially the admixture contains one or more neutral fillers.

It was surprisingly found that other common pharmaceutical fillers were not as effective in attenuating the degradation of the compositions as basic particulate solids. In particular, it was found that mannitol and calcium hydrogen phosphate (e.g. anhydrous) had a detrimental effect on the stability of the compositions. Preferably, the composition, and in particular the admixture comprising the compound of Formula I or salt or ester or solvate thereof, is substantially free of mannitol, sucrose, glucose, dextrose, lactose, xylitol, fructose, sorbitol, calcium phosphate and/or calcium sulphate. In certain embodiments, the composition of the invention is substantially free of microcrystalline cellulose; in other embodiments, the composition may contain microcrystalline cellulose. For example, the fillers in the admixture may comprise 10-20% weight of basic particulate solid and 80-90% weight microcrystalline cellulose.

The term “substantially free” as used herein when referring to a particular constituent of the admixture means the admixture contains no more than 5% by weight of the constituent based on the total weight of the admixture, preferably no more than 2% by weight, more preferably no more than 1% by weight and especially no more than 0.3% by weight of the constituent based on the total weight of the admixture. For example, the admixture may have no more than 0.1% by weight by weight of the constituent based on the total weight of the admixture and in some embodiments the admixture may have no more than 0.01% by weight of the constituent based on the total weight of the admixture.

In addition to the compound of Formula I or salt or ester or solvate thereof and the basic particulate solid, the admixture of the compositions of the invention may comprise one or more further pharmaceutical excipients. Preferred further pharmaceutical excipients should not have a detrimental effect on the stability of the compound of Formula I or salt or ester or solvate thereof. The further pharmaceutical excipients are preferably neutral or basic. The one or more further pharmaceutical excipients may comprise one or more of a binder, a disintegrant and a lubricant. Exemplary binders include maltodextrin. Preferably, the binder is maltodextrin. Exemplary disintegrants include salts of carboxymethyl cellulose (the sodium salt being available commercially as Croscarmellose sodium), carboxymethyl starch derivatives (such as the sodium salt that is available commercially as Primojel), and starch (e.g. the starch available commercially as Maydis amylum). Preferably, the disintegrant is carboxymethyl cellulose, a carboxymethyl starch derivatives or starch. Preferably, the lubricant is magnesium stearate. The presence of one or more of maltodextrin, Croscarmellose sodium, Primojel, Maydis amylum and magnesium stearate in the composition has not been found to have a detrimental effect on the stability of the compound of Formula I. In one embodiment the composition comprises an admixture of a compound of Formula I, calcium carbonate, maltodextrin, carboxymethylcellulose and magnesium stearate.

Ingredients which may in some embodiments be present in the admixture of the present invention, but which in other embodiments of the invention are preferably absent from the admixture, include cellulose ethers (e.g. the 2-hydroxypropylmethyl ether of starch available under the trade mark Hypromellose 6); mannitol; cellulose; lactose monohydrate; calcium hydrogen phosphate anhydride; dibasic calcium phosphate dehydrate; starch for example pregelatinised starch; gelatine; silica; polyethylene glycol; polyvinyl acetate phthalate; and/or triethyl citrate. Some acidic excipients have been found to have a detrimental effect on the stability of the compositions when admixed with compounds of Formula I. The admixture may be substantially free of acidic excipients.

The compositions may be formulated in a process in which the active ingredients are mixed with a solvent. Traces of solvent may be present in the compositions.

In certain embodiments of the first aspect of the invention, the composition may include an antioxidant. Suitable antioxidants include, for example, sodium ascorbate. The presence of acidic antioxidants may however be detrimental to the stability of the composition. Therefore, in some embodiments of the invention, the admixture comprising the compound of Formula I or salt or ester or solvate thereof is substantially free of an antioxidant, especially an acidic antioxidant.

In a further, preferred, embodiment of the invention, the composition is formed into a solid dosage form (e.g. a tablet or capsule) that comprises a core and a coating. Preferably, the dosage form is a tablet. In one embodiment the composition is a coated tablet wherein the admixture of components (i), (ii) and optional components (iii) are present in the core of the tablet. Component (i) may be milled either before or after being admixed with the pharmaceutically acceptable basic particulate solid.

It has been found that the presence of various excipients can be tolerated in the compositions when those compounds are not admixed with the compound of Formula I or salt or ester or solvate thereof in the core of the dosage form. For example, it has been found that when various excipients are present as a constituent of the coating they do not have a detrimental effect on the stability of compounds of Formula I present in the core. For example, triethyl citrate may be present in a coating without adversely affecting the stability of the composition whereas the presence of triethyl citrate in the core of a tablet may enhance the degradation of the composition. The coating may, for example, comprise one or more of methacrylic acid-ethyl acrylate copolymer (1:1), talc (magnesium silicate), triethyl citrate and Opadry® AMB (a coating material available from Colorcon, PA, USA). The presence of stearic acid, Opadry AMB, citric acid and talc in coatings of compositions comprising a core having a compound of Formula I has not been found to be detrimental to stability.

In one embodiment, the composition is provided with an enteric coating. Preferably, the composition is an enteric coated tablet. The enteric coating is preferably formed using any commercially-available polymer produced for such a purpose. As examples of such polymers, those based on acrylates, methacrylates or copolymers thereof (such as the range of enteric coating polymers marketed under the name Eudragit® by Degussa/Roehm), polyvinyl acetate phthalate, cellulose acetate phthalate, hydroxypropylmethylcellulose acetate succinate, hydroxypropylmethylcellulose phthalate, hydroxymethylcellulose acetate succinate and carboxymethylethylcellulose may be mentioned. Different coatings which dissolve at different pH ranges may be used for different applications. In a particular embodiment, the enteric coating comprises a methacrylic acid-ethyl acrylate copolymer. The constituent monomers of such a copolymer may be present in the ratio 1:1. The enteric coating also preferably contains a glidant component, such as talc. A plasticiser may also be advantageously included. A suitable plasticiser is triethyl citrate.

In most circumstances, it may be preferable for an inert coating to be provided between that portion of the composition containing the compound of Formula I or salt or ester or solvate thereof and the enteric coating. Preferably, an inert coating is provided between the core of a dosage form and the enteric coating. Enteric coatings are typically composed of acidic polymers and hence, by their very nature, have the potential to lead to deleterious changes in certain active ingredients. Similarly, a basic tablet core can lead to deleterious changes in an enteric coating. An interposed inert coating (made from, for example, a cellulose derivative, such as hydroxypropyl cellulose or hydroxypropylmethyl cellulose) tends to inhibit such interactions. An inert coating is one which is resistant to reaction with ingredients of both the tablet core and the enteric coating. The inert coat should, of course, be soluble (or otherwise dispersible) in the intestinal media in order to allow the active ingredient to be released. Suitable coating materials include PVA-PEG graft copolymers; cellulose ethers; hypromellose phthalate; cellulose acetate phthalate; hypromellose; maltodextrin; polydextrosepolyvinylpyrrolidone; and shellac. Commercially-available materials suitable for use as an inert coat include for example those marketed under the trade marks Aquacoat AS-LG (available from FMC), Kollicoat IR (available from BASF) and Sepifilm.

The term ‘enteric coating’ as used herein is intended to include coatings applied to a composition/dosage form once the dosage form is otherwise essentially complete and those applied at intermediate stages of dosage form manufacture. Thus, compositions are included in which the compound of Formula I or salt or ester or solvate thereof is formulated with excipients into granules which are then enteric coated prior to further processing, such as compression into tablets or filling into capsules, for example gelatin capsules. Those skilled in the art will appreciate that the term “enteric coating” further intends to specify a coating which displays specific disintegration and dissolution properties. The present invention specifically concerns such enteric coatings which have dissolution properties which efficiently protect the tablet core ingredients, such as the compound of Formula I, from exposure to the acidic environment of the GI tract. Coatings which dissolve or otherwise disintegrate or disperse only at pH values in excess of 5 are preferred. Such coatings may dissolve at pH values above 5.5, such as above pH 6, or above pH 6.5. Mixtures of different grades and makes of such enteric coatings are included in the invention.

According to a second aspect, the invention provides a method of preparing a pharmaceutical composition suitable for oral administration, comprising the step of admixing a compound of Formula I or salt or ester or solvate thereof and a pharmaceutically acceptable basic particulate solid. Further pharmaceutical excipients may also be admixed with these ingredients. The method may comprise the step of milling the Compound of Formula I or salt or ester or solvate thereof. The method may comprise the step of milling the admixture, or may comprise the step of milling the Compound of Formula I or salt or ester or solvate thereof prior to admixing it with the pharmaceutically acceptable basic particulate solid. The method may include the step of granulating the admixture. The method may comprise the step of providing the granules with a coating (e.g. an inert coating or an enteric coating). The method may further comprise the step of processing the admixture into a solid unit dosage form. The processing of the admixture into the solid unit dosage form may include the step of granulating the admixture. The admixture or granules may be formed into a solid unit dosage form (e.g. a tablet) core. The method may comprise the step of providing the solid unit dosage form core with a coating. Preferably, the method includes the step of providing the solid unit dosage form core with an inert coating. Preferably the method includes the step of providing the solid unit dosage form core with an enteric coating.

The invention further provides a method of preparing a pharmaceutical composition suitable for oral administration, comprising (i) a compound of Formula I or a pharmaceutically acceptable salt or ester or solvate thereof and (ii) a pharmaceutically acceptable basic particulate solid comprising the steps of:
a. optionally mixing the pharmaceutically acceptable basic particulate solid and a binder;
b. dissolving component (i) in a solvent;
c. mixing the solution of component (i) in a solvent from step (b) with component (ii) that has optionally been mixed with the binder in optional step (a) to form a wet mass;
d. optionally granulating the wet mass from step (c);
e. drying the wet mass from step (c) or granulate from optional step (d);
f. optionally grinding or milling or sieving the mixture;
g. optionally adding a disintegrant and mixing;
h. optionally adding a lubricant and mixing;
i. forming the mixture into tablets;
j. optionally coating the tablets with an inert coating; and
k. coating the tablets with an enteric coating.

In some preferred compounds of Formula I, R₁ is isopropyl, iodo or H. In addition or alternatively, it may be preferred that R₂ and R₃ are each independently halogen or alkyl. In such a case, R₂ and R₃ are preferably each independently Cl, Br, I or methyl. In certain preferred embodiments, R₂=R₃. R₂=R₃=Br or Cl are especially preferred. R₄ is preferably H or methyl, with H particularly preferred. R₅ is preferably H.

The term “alkanoyl” as employed herein alone or as part of another group is alkyl linked to a carbonyl group. The term “aroyl” as employed herein alone or as part of another group is aryl linked to a carbonyl group. Unless otherwise indicated, the term “alkyl” or “alk” as employed herein alone or as part of another group includes both straight and branched chain hydrocarbons, containing 1 to 12 carbons in the chain, preferably 1 to 4 carbons. The term “aryl” as employed herein alone or as part of another group refers to monocyclic and bicyclic aromatic groups containing 6 to 10 carbons in the ring portion and may be optionally substituted through available carbon atoms with 1, 2, or 3 groups selected from hydrogen, halo, alkyl, haloalkyl, alkoxy, haloalkoxy, alkenyl, trifluoromethyl, trifluoromethoxy, alkynyl, hydroxy, amino, nitro, cyano and/or carboxyl or alkyl ester thereof.

When a compound of Formula I is present in the form of an ester, an alkyl ester thereof is preferred, especially a C₁₋₄ alkyl ester.

Preferably the compound of Formula I is the free acid or a salt, especially the calcium salt. It is believed that the calcium salt of compounds of Formula I, such as the calcium salt of Compound 1A, are novel. The invention therefore provides said calcium salts per se, and also provides a pharmaceutical composition suitable for oral administration, comprising the calcium salt of a compound of Formula I, especially the calcium salt of the compound of Formula IA, together with one or more pharmaceutical excipients.

When a compound of Formula I is present in the form of a pharmaceutically acceptable salt, such salts may include metal salts, such as alkali metal or alkaline earth metal salts, for example sodium, potassium, calcium or magnesium salts, or salts with ammonia or an organic amine, such as morpholine, thiomorpholine, piperidine, pyrrolidine, a mono, di or tri-lower alkylamine, for example ethyl, tertbutyl, diethyl, diisopropyl, triethyl, tributyl or dimethylpropylamine, or a mono, di or trihydroxy lower alkylamine, for example mono, di or triethanolamine. Preferred salts of the compounds of Formula I include sodium, potassium, calcium and magnesium salts and salts with pharmaceutically acceptable organic amines. The calcium salt is especially preferred. The calcium salt of Compound 1A has been found to be particularly resistant to degradation.

The compounds of Formula I may of course be solvated if desired, for example hydrates may be used in the present invention.

The present invention also provides a composition according to the invention, for use in therapy.

The present invention also provides the use of a composition according to the invention in the preparation of a medicament for preventing, inhibiting or treating a disease associated with metabolism dysfunction, or which is dependent on the expression of a triiodothyronine (T₃)-regulated gene. The disease may be selected from obesity, hypercholesterolemia, dyslipidaemia, atherosclerosis, cardiac arrhythmias, depression, osteoporosis, hypothyroidism, goitre, thyroid cancer as well as glaucoma and congestive heart failure. Similarly, the present invention also provides a method of preventing, inhibiting or treating a disease associated with metabolism dysfunction, or which is dependent on the expression of a triiodothyronine (T₃)-regulated gene, the method involving the administration of a composition according to the invention to a subject in need of such prevention, inhibition or treatment.

In the use and method described immediately above, the medicament or composition may be administered at a dosing interval of from 30 minutes to one month. More preferably, the dosing interval is from one to seven days, even more preferably one to three days. The typical adult human dose range for compounds (i) would be around 1 μg to around 2000 μg per day. For many compounds (i), the daily dose would be less than 300 μg. Preferably, the dose of compound (i) is from around 1 μg to around 200 μg per day, more preferably around 1 to around 100 μg per day. For example, the compounds (i) may be administered in one dose, two doses, three doses or four doses per day. Preferably, the amount of compound (i) per unit dose of composition is from 1 to 200 μg, more preferably 1 to 100 μg, more preferably 1, 5, 10, 20, 25 or 50 μg. For example, the amount of compound (i) per unit dose may be from 10 to 100, for example from 20 to 80, typically from 25 to 50, μg.

The composition according to the invention may also comprise a further pharmacologically active ingredient selected from hypolipidaemic agents, including statins, for example atorvastatin or simvastatin, antidiabetic agents, antidepressants, bone resorption inhibitors, appetite suppressants and/or anti-obesity agents. In a particularly preferred embodiment, the composition according to the invention may also comprise the anti-hyperlipidaemic agent ezetimibe.

The further pharmacologically active ingredients tend to have additive or synergistic effects with the compounds of Formula I or salts or esters or solvates thereof so as to enhance the metabolic effects thereof. In yet another aspect, the present invention provides a combination medicament suitable for oral administration, comprising:

(1) a first pharmaceutical composition comprising a compound of Formula I or a pharmaceutically acceptable salt or ester or solvate thereof, in admixture with a pharmaceutically acceptable basic particulate solid and as described above; and

(2) a second pharmaceutical composition comprising at least one antioxidant, wherein the second pharmaceutical composition contains at least one pharmaceutically-acceptable excipient, and wherein the first and second pharmaceutical compositions may be administered simultaneously, sequentially or separately.

The combination medicament of the present invention takes advantage of the fact that, as described in WO 2007/110226, it is desirable that an antioxidant should be present when the compound of the formula I or a salt or ester or solvate comes into contact with acid and a source of nitrite. Provided the two compositions of the combination medicament are given in such temporal proximity that there is overlap between their periods of residence in the stomach, the active compound should enjoy at least a degree of stabilisation against the nitration reaction.

The invention will now be described in more detail by way of example only and with reference to the appended drawings, of which:

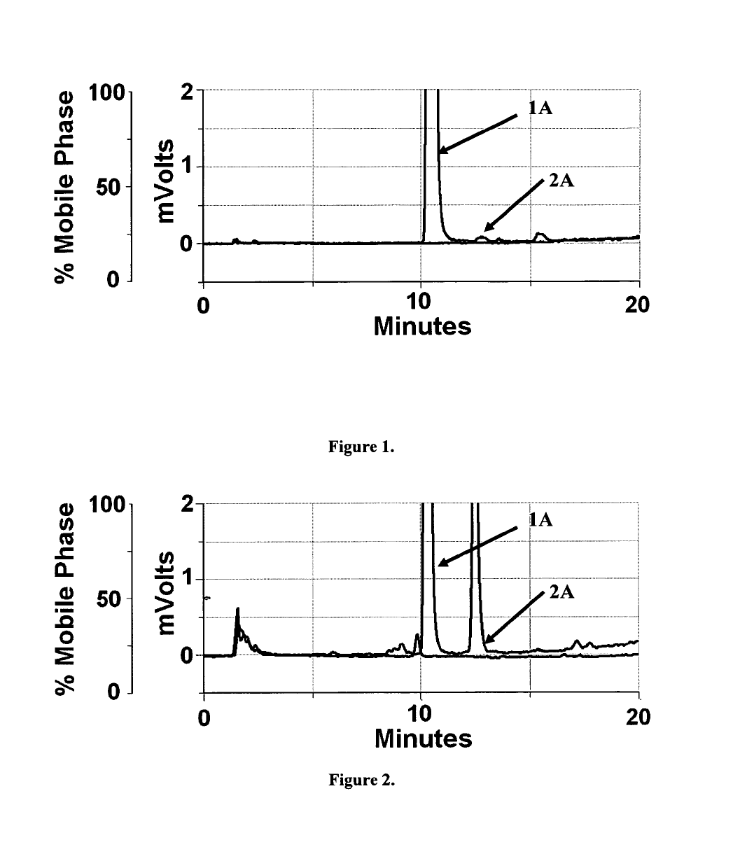

FIG. 1 shows an HPLC trace of freshly prepared sample of Compound 1A;

FIG. 2 shows an HPLC trace of sample 1 of Example 1 after storage for 4 weeks;

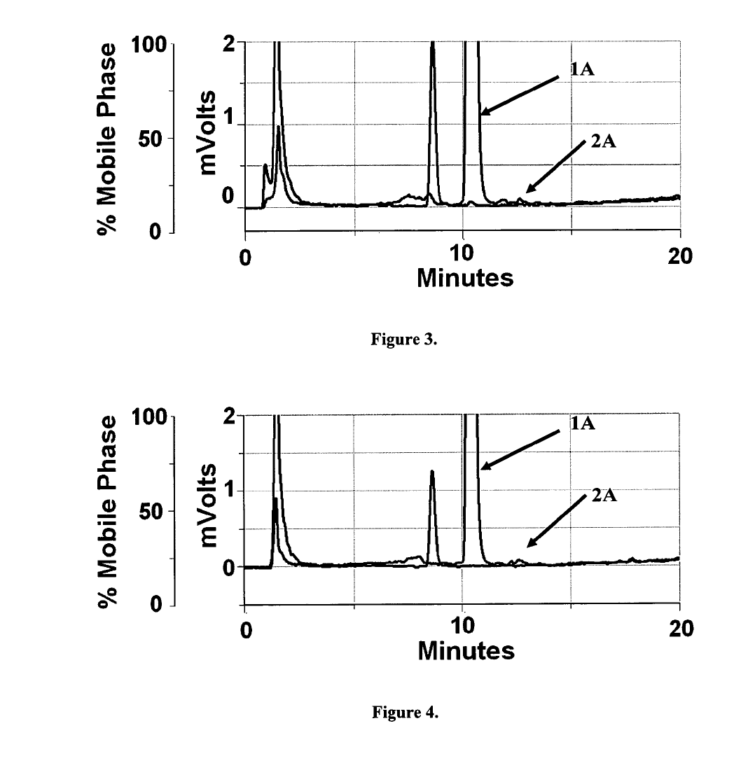

FIG. 3 shows an HPLC trace of sample 2 of Example 1 after storage for 4 weeks;

FIG. 4 shows an HPLC trace of sample 3 of Example 1 after storage for 4 weeks;

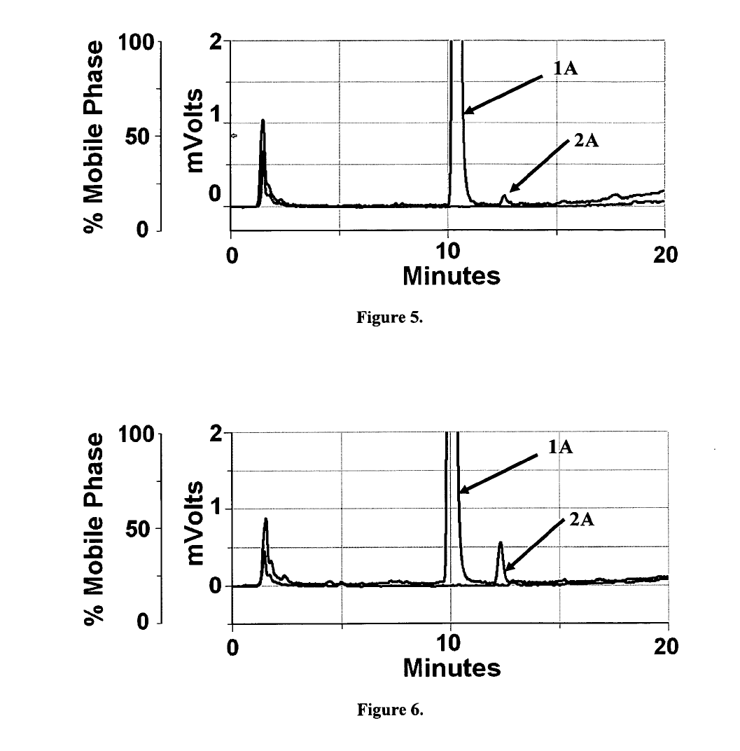

FIG. 5 shows an HPLC trace of sample 4 of Example 1 after storage for 4 weeks;

FIG. 6 shows an HPLC trace of sample 5 of Example 1 after storage for 4 weeks;

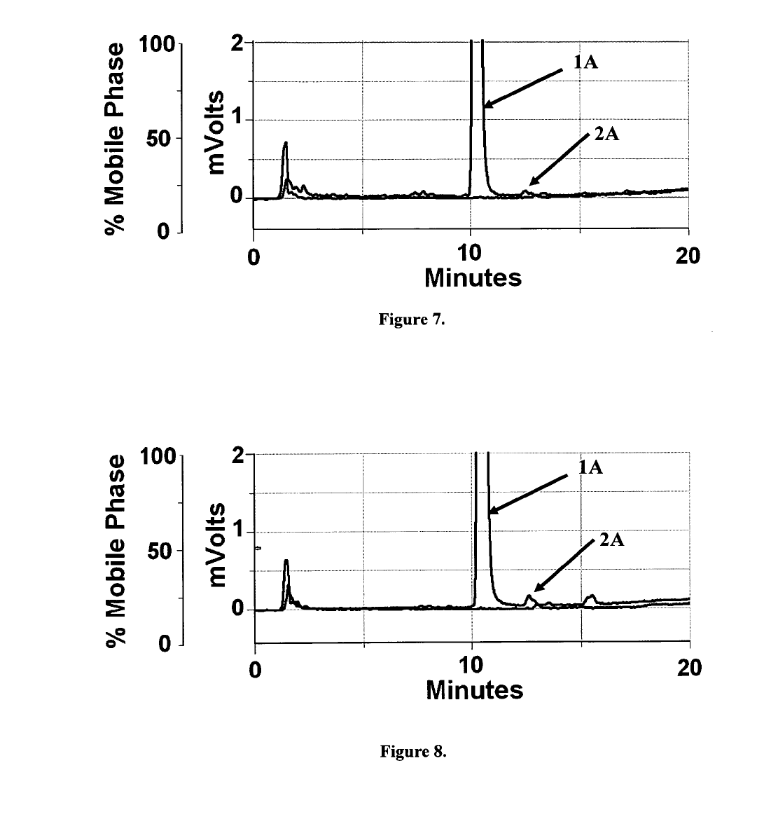

FIG. 7 shows an HPLC trace of sample 6 of Example 1 after storage for 4 weeks;

FIG. 8 shows an HPLC trace of sample 7 of Example 1 after storage for 4 weeks;

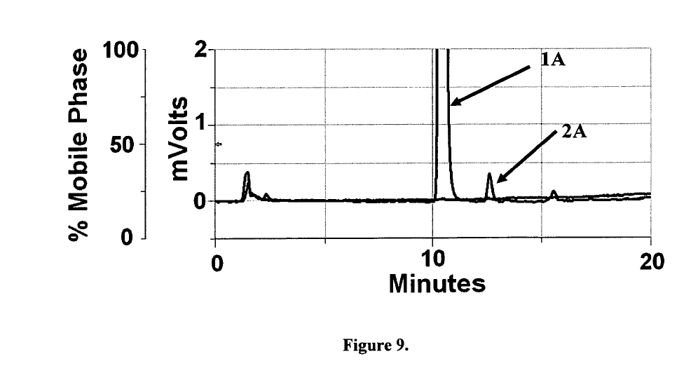

FIG. 9 shows an HPLC trace of sample 8 of Example 1 after storage for 4 weeks.

The following materials are used in the Examples and/or are referred to above:

Croscarmellose sodium=CAS 74811-65-7, Cellulose, carboxymethyl ether, sodium salt, crosslinked
Primojel=CAS 9063-38-1, Sodium carboxymethyl starch
Maydis amylum=CAS 9005-25-8, Starch
Maltodextrin=CAS 9050-36-6, maltodextrin
Magnesium stearate=CAS 557-04-0, Octadecanoic acid magnesium
Hypromellose=CAS 9004-65-3, Cellulose, 2-hydroxypropyl methyl ether

### Mannitol=CAS 69-65-8, D-Mannitol

Cellulosum microcrist=CAS 9004-34-6, Cellulose
Lactose monohydrate=CAS 64044-51-5, O-β-D-Galactopyranosyl-(1→4)-α-D-glucopyranose monohydrate, the anhydrous form being CAS 63-42-3 O-β-D-Galactopyranosyl-(1→4)-α-D-glucopyranose anhydrous
Pregel starch=CAS 9005-25-8, Pregelatinized starch
Opadry AMB=a water soluble coating material available from Colorcon, Pa., USA
Eudragit L30-D50=a methacrylic acid-ethyl acrylate copolymer (1:1) that is available from Degussa/Roehm GmbH & Co. KG of Darmstadt, Germany Ready having a Ph of 2.1-3.0 determined according Ph. Eur. 2.2.3.

### EXAMPLE 1

### Compatibly of Compound IA when Mixed with Various Excipients

### 1.1—Introduction

The aim of this study is to demonstrate the stability of the compound of Formula IA when dissolved in a solvent and mixed with various excipient blends.

### 1.2—Sample Preparation

Comparative sample 1 and samples 2 to 6 were prepared by dissolving an unmilled 1 mg sample of Compound 1A in a 1:1 mixture of ethanol and water, adding the solution to the a dry mixture of the excipients listed in Table 1 and mixing with a spatula. Sample 7 was prepared by dissolving a milled 1 mg sample of Compound 1A in a 1:1 mixture of ethanol and water, adding the solution to the a dry mixture of the excipients listed in Table 1 and mixing with a spatula.

Sample 8 was prepared by dissolving an unmilled 1 mg sample of Compound 1A in ethanol, adding the solution to the a dry mixture of the excipients listed in Table 1 and mixing with a spatula.

TABLE 1
Guidance amounts of excipients in the granulate compatibility study

| | | | Cell- | | | | | | | |
|---|---|---|---|---|---|---|---|---|---|---|
| | | Hypro- | losum | Malto- | Ca | Cros- | | Maydus | Starch pre- | |
| | Solvent | mellose | microcr. | dextrin | carbonate | carmellose | Primojel | amylum | gelatinised | Granulate¹ |
| No. | (μl) | (mg) | (mg) | (mg) | (mg) | (mg) | (mg) | (mg) | (mg) | (mg) |
| 1 | 280 | 7 | 1579 | | | | | | | |
| 2 | 280 | | | 111 | 1092 | 140 | | | | |
| 3 | 280 | | | 111 | 1092 | | 140 | | | |
| 4 | 280 | | | 111 | 1092 | | | 140 | | |
| 5 | 280 | | | 111 | 1092 | | | | 140 | |
| 6 | 280 | | | | | | | | | 1300 |
| 7 | 0² | | | | | | | | | 1300 |
| 8 | 280³ | | | | | | | | | 1300 |

¹Granulate contains calcium carbonate with 9% maltodextrin.
²Dry mixture with milled Compound 1A.
³Compound 1A dissolved in 280 μl ethanol, no water added.

Samples 1 to 8 were dried in a tray drier at 40° C. overnight to remove the liquid and then stored in open vials at 50° C. and 75% relative humidity. The samples were then removed after two or four weeks and stored at room temperature for up to four days prior to being analysed.

The blends of Compound 1A and excipients were transferred to a glass volumetric flask and diluted to a volume of 10 ml with a 0.05% trifluoroacetic acid (TFA) solution in 50/50 acetonitrile/water to provide a 1 mg/ml solution of Compound 1A. The solution was stirred using a magnetic stirrer for 20 minutes then left for 5 minutes before 1 ml aliquots were taken using a plastic syringe. The aliquots were filtered through a 0.45 μm syringe filter int amber glass vials for HPLC analysis.

### 1.3—Results

Results from HPLC analysis of samples stored for 2 and 4 weeks are shown in Table 2 below. The primary degradation product detected is Compound 2A and therefore the area of the trace for degradation product Compound 2A was compared with the area of the trace for compound 1A. Compound 2A is the acetamide formed when Compound 1A is decarboxylated.

TABLE 2
Results from the granulate compatibility study with Compound 1A.

| | | Area % | |
|---|---|---|---|
| | | Compound 2A/ | |
| | | Compound 1A¹ | |
| Sample | | 2 | 4 |
| No. | Excipients in granulate blend | weeks | weeks |
| 1 | Hypromellose + cellulosum microcrist (Comparative) | 10.0 | 21 |
| 2 | Maltodextrin + calcium carbonate + croscarmellose | 0.2 | 0.2 |
| 3 | Maltodextrin + calcium carbonate + primojel | 0.2 | 0.2 |
| 4 | Maltodextrin + calcium carbonate + maydis amylum | 0.2 | 0.3 |
| 5 | Maltodextrin + calcium carbonate + pregel starch | 0.8 | 1.4 |
| 6 | Granulate with calcium carbonate and 9% maltodextrin | 0.1 | 0.2 |
| 7 | Granulate with calcium carbonate and 9% maltodextrin- dry mixture² | 0.1 | 0.2 |
| 8 | Granulate with calc. carbonate and 9% maltodextrin, Compound 1A in 100 | 0.9 | 1.4 |
| | % EtOH | | |

¹Initial concentration of Compound 1A is 0.2%
²1 mg milled Compound 1A added to the mixture

In order for the final drug product to be acceptable, the concentration of degradation Compound 2A should be below or equal to 2.0 area % of Compound 1A and especially below or equal to 1.5 area % of Compound 1A.

Samples 2, 3, 4 and 6 in which Compound 1A was dissolved in ethanol/water 1:1 and mixed with the “wet” blends showed particularly low levels of degradation. Comparative sample 1 demonstrated that Compound 1A was not stable in combination with hypromellose and cellulosum microcrist.

### 1.5—Conclusions

The results from this study show that Compound 1A is stable when dissolved in ethanol/water 1:1 and granulated with calcium carbonate.

The presence of a pharmaceutically acceptable basic particulate solid in admixture with the compound of Formula 1A may provide conditions under which decarboxylation to form Compound 2A is less favourable. It is also speculated that the basic particulate solid, and calcium carbonate in particular, may affect the crystal structure of the compound of Formula 1A resulting in a form in which decarboxylation reactions are less favoured.

### EXAMPLE 2

### Stability of Compound 1A when Milled

### 2.1—Introduction

Investigate stability of milled and unmilled Compound 1A in combination with calcium carbonate.

### 2.2—Sample Preparation

Samples containing 1092 mg of calcium carbonate and 1 of Compound 1A (either milled or unmilled) were prepared and weighed into small amber glass bottles. Samples were stored in open vials at 50° C. and 75% relative humidity. The samples were then removed after two or four weeks and stored at room temperature for up to four days prior to being analysed and were stored in open vials at 50° C. and 75% RH. The blends of Compound 1A and calcium carbonate were transferred to a glass volumetric flask and diluted to a volume of 10 ml or 100 ml with a 0.05% trifluoroacetic acid (TFA) solution in 50/50 acetonitrile/water to provide a 1 mg/ml solution of Compound 1A. The solution was stirred using a magnetic stirrer for 20 minutes then left for 5 minutes before 1 ml aliquots were taken using a plastic syringe. The aliquots were filtered through a 0.45 μm syringe filter into amber glass vials for HPLC analysis.

### 2.3—Results

No difference in stability has been detected between samples of Compound 1A mixed with calcium carbonate that were milled and unmilled either initially or after storage in open vials at 50° C. and 75% relative humidity for 2 or 4 weeks. The area % of Compound 2A compared to Compound 1A remained at the initial concentration of 0.2 in both milled and unmilled samples after 2 and 4 weeks.

### EXAMPLE 3

### Enterically Coated Tablets Comprising Compound 1A

Tablet cores comprising 25 or 50 μg of Compound 1A in admixture with calcium carbonate were successfully provided with an enteric coating comprising a Methacrylic acid-ethyl acrylate copolymer using a coating pan. No discoloration of the enterically coated tablets was observed on storage at room temperature for four weeks.

### EXAMPLE 4

### Enteric-Coated Tablets with an Interposed Inert Layer

In this example, all quantities are given in mg per dosage unit. Calcium carbonate (filler, 122.8) and maltodextrin (binder, 12.15) were dry mixed in a high shear mixer. Compound 1A (0.025) was dissolved, using a mixer, in a blend of purified water (15) and ethanol (10), and the solution was added with agitation to the mixture of calcium carbonate and maltodextrin. The resulting wet mass was granulated, dried using a fluid bed drier, and sieved through a mill. The sieved granulate was then mixed with croscarmellose sodium (disintegrant) (4.2) using a double-cone blender, and the resulting mixture mixed together with magnesium stearate (lubricant, 0.85) in a double-cone blender. The resulting powder blend was compressed into tablets using a rotary tablet press.

An inert coating of Opadry AMB® (6.0) was applied to the resulting tablet cores using a coating pan. A further, enteric, coating was applied by using a 30% dispersion of Eudragit L30-D55®, a 1:1 methacrylic acid-ethyl acrylate copolymer (25.0), together with talc (glidant, 7.0) and triethyl citrate (plasticizer, 1.4) in a coating pan.

A second batch of tablets was prepared in exactly the same way, save that the content of Compound 1A was 0.050 mg/unit.

Satisfactory stable tablets were obtained.

Long-term storage tests were carried out on the enteric coated tablets containing 50 μg of Compound 1A. Normal variation of the initial content of Compound 1A is 47.5-52.5. After storage for 9 months at 30° C. and 65% relative humidity, the measured content of Compound 1A was 48.8. No change in the visual appearance of the tablets was observed.

1. A pharmaceutical composition suitable for oral administration, comprising an admixture of:
(i) a compound of Formula I: 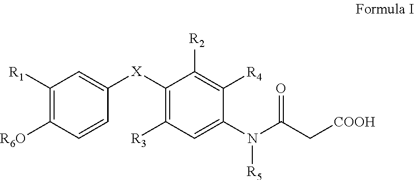
wherein:
R₁ is selected from hydrogen, halogen, trifluoromethyl, C₁₋₆ alkyl or C₃₋₇ cycloalkyl;
X is oxygen (—O—), or methylene (—CH₂—);
R₂ and R₃ are the same or different and are halogen or C₁₋₄ alkyl;
R₄ is hydrogen or C₁₋₄ alkyl;
R₅ is hydrogen or C₁₋₄ alkyl; and
R₆ is hydrogen, or an alkanoyl or aroyl group, or other group capable of bioconversion to generate the free phenol structure wherein R₆=H;
or a pharmaceutically acceptable salt or ester or solvate thereof;
(ii) a pharmaceutically acceptable basic particulate solid; and
(iii) optionally one or more further pharmaceutical excipients,
wherein component (ii) makes up at least 1% by weight of the admixture.

2. The composition according to claim 1, wherein component (ii) makes up at least 40% by weight of the admixture.

3. The composition according to claim 1, in which component (i) is 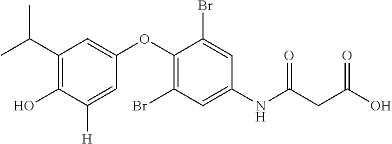
or a pharmaceutically acceptable salt or ester or solvate thereof.

4. The composition accordingly to claim 1 wherein component (ii) is a Group 1 or Group 2 metal carbonate or bicarbonate.

5. The composition according to claim 4, wherein component (ii) is calcium carbonate.

6. The composition accordingly to claim 1, wherein pharmaceutically acceptable basic particulate solids make up at least 70% by weight of the admixture.

7. The composition according to claim 1, wherein the admixture is substantially free of any fillers other than basic particulate solids.

8. The composition according to claim 1 wherein component (i) is milled.

9. The composition according to claim 1, wherein component (iii) comprises one or more of a binder, a disintegrant and a lubricant.

10. The composition according to claim 9 wherein the binder is maltodextrin; the disintegrant is a carboxymethylated cellulose, a carboxymethyl starch or a starch; and/or the lubricant is magnesium stearate.

11. The composition according to claim 1, comprising a core and a coating wherein the admixture of components (i) and (ii) and optional component(s) (iii) is present in the core.

12. The composition according to claim 10 further comprising an enteric coating.

13. The composition according to claim 11, further comprising an inert coating interposed between the core and the enteric coating.

14. The composition according to claim 1, further comprising a pharmacologically active ingredient selected from hypolipidaemic agents, antidiabetic agents, antidepressants, bone resorption inhibitors, appetite suppressants and anti-obesity agents.

15. A method of preparing a pharmaceutical composition suitable for oral administration, comprising admixing
(i) a compound of Formula I: 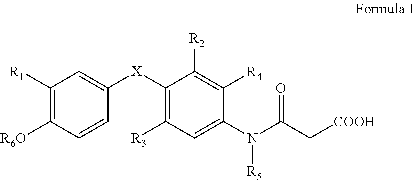
wherein:
R₁ is selected from hydrogen, halogen, trifluoromethyl, C₁₋₆ alkyl or C₃₋₇ cycloalkyl;
X is oxygen (—O—), or methylene (—CH₂—);
R₂ and R₃ are the same or different and are halogen or C₁₋₄ alkyl;
R₄ is hydrogen or C₁₋₄ alkyl;
R₅ is hydrogen or C₁₋₄ alkyl; and
R₆ is hydrogen, or an alkanoyl or aroyl group, or other group capable of bioconversion to generate the free phenol structure wherein R₆=H;
or a pharmaceutically acceptable salt or ester or solvate thereof; and
(ii) a pharmaceutically acceptable basic particulate solid,
wherein component (ii) makes up at least 1% by weight of the admixture.

16. The method according to claim 15 further comprising the step of forming the admixture of (i) and (ii) into a tablet core

17. The method according to claim 16 further comprising the step of providing the tablet core with an enteric coating.

18. The method according to claim 15, further comprising the step of milling component (i).

19. The method according to claim 15 further comprising the steps of:
a. optionally mixing a pharmaceutically acceptable basic particulate solid and a binder;
b. dissolving component (i) in a solvent;
c. mixing the solution of component (i) in a solvent from step (b) with component (ii) that has optionally been mixed with the binder in optional step (a) to form a wet mass;
d. optionally granulating the wet mass from step (c);
e. drying the wet mass from step (c) or granulate from optional step (d);
f. optionally milling the mixture;
g. optionally adding a disintegrant and mixing;
h. optionally adding a lubricant and mixing;
i. forming the mixture into tablets;
j. optionally coating the tablets with an inert coating; and
k. coating the tablets with an enteric coating.

20. (canceled)

21. A method of preventing, inhibiting or treating a disease associated with metabolism dysfunction, or which is dependent on the expression of a triiodothyronine (T₃)-regulated gene comprising administering a composition according to claim 1.
